(19) United States
(12) Patent Application Publication
(10) Pub. No.: US 2002/0151051 A1
(43) Pub. Date: Oct. 17, 2002

(54) Compositions and methods for isolating genes comprising subcellular localization sequences

(76) Inventor: Sheng Feng Li, Belmont, CA (US)

Correspondence Address: HOWREY SIMON ARNOLD & WHITE, LLP, BOX 34, 301 RAVENSWOOD AVE., MENLO PARK, CA, 94025, US

(21) Appl. No.: 10/108,118

(22) Filed: Mar. 26, 2002

Related U.S. Application Data
(60) Provisional application No. 60/279,258, filed on Mar. 27, 2001.

(51) Int. Cl. C12N 5/06; C12N 15/00; C07H 21/04

(52) U.S. Cl. 435/325; 435/320.1; 536/23.2

(57) ABSTRACT

The present invention provides an expression vector and library thereof suited for categorizing and identifying genes comprising subcellular localization sequences. The invention vectors are particularly suited for isolating extracellular membrane bound, extracellular or secreted proteins. The present invention also provides kits and eukaryotic host cells comprising the invention vectors. Further provided by the invention are methods of using the subject vectors for cloning genes encoding proteins that are preferentially located in certain subcellular locations. Also included is a method of determining the subcellular location of a protein.

### CROSS-REFERENCE TO RELATED APPLICATIONS

This application claims the priority benefit of U.S. Provisional Patent Application 60/279,258, filed Mar. 27, 2001, pending, which is hereby incorporated herein by reference in its entirety.

### TECHNICAL FIELD

This invention is in the field of genetic analysis. Specifically, the invention relates to the generation of expression vectors and libraries thereof that allow classification and identification of genes based on the subcellular localization patterns of the encoded protein products. The compositions and methods embodied in the present invention are particularly useful for isolating genes encoding membrane bound, extracellular, and nuclear proteins.

### BACKGROUND OF THE INVENTION

The rapid advancement in genomics studies within the past five years begins a new era for biological research. To date, more than twenty prokaryotic genomes have been delineated, several eukaryotic genomes including yeast (S. cerevisia), nematode (C. elegance), fruitfly (Drosophila melanogaster), and even the human genome have been sequenced. With the imminent refinement of the entire human genome sequences and the completion of that of other organisms, the next objective is to harness this vast wealth of genetic information in the prediction, diagnosis and treatment of diseases. Such a venture requires an understanding of the biological functions of the sequenced genes. Elucidation of the biological functions of a gene often involves determining the subcellular expression pattern of the encoded protein product.

Unlike a prokaryotic cell which generally consists of a single compartment surrounded by a plasma membrane, a eukaryotic cell is elaborately subdivided into functionally distinct, membrane-bounded compartments. Each compartment, or organelle, contains its own distinct set of proteins and other specialized molecules. A complex distribution system conveys specific products from one compartment to another. A mammalian cell contains approximately 10 billion protein molecules of perhaps more than 30,000 kinds (excluding the immunoglobulins which are estimated to be 10⁹ to 10 ¹²/per cell), and the synthesis of almost all of these begins in the cytosol, the common space that surrounds the organelles. Each newly synthesized protein is then delivered specifically to the cellular compartment requiring the protein.

The delivery and confinement of proteins to specific subcellular locations are critical for maintaining cell function. Perturbations of the intracellular protein trafficking events have long been acknowledged to lead to aberrant behavior of a disease cell. Abnormal subcellular expression patterns, in form of retention of proteins in organelles in which they do not normally reside, secretion of otherwise cytosolic proteins, or delivery of otherwise cytosolic proteins to the nucleus or the plasma membrane, account for a vast number of abnormal cellular responses. Among them are cell transformation, metastasis, unscheduled differentiation, and apoptosis.

Traditional methods for determining the subcellular location of a protein are largely restricted to subcellular fractionation, cytoimmuno-staining, and electron microscopy. These techniques not only require prior knowledge of the protein that is to be examined but also have pronounced disadvantages. For instance, cell fractionation generally yields a partial separation of some and not all individual cellular organelles (see, e.g. an exemplary fractionation system, the hybrid Percoll/metrizamide discontinuous density gradient as described in (Storrie, et al. (1990) Methods Enzymol 182:203-225). Cytoimmuno-staining is applicable only when a highly specific antibody reactive with the target protein is available. Whereas electron microscopy can track the subcellular distribution of a protein under high resolution, the method is extremely costly, time consuming and certainly not amenable for high throughput analysis. Thus, there remains a considerable need for compositions and methods to effect a more robust subcellular localization analysis.

Likewise, conventional procedures for isolating genes encoding proteins that are localized to particular cellular compartments are limited to traditional screening assays and expression cloning techniques. Both procedures require some sequence information of the target gene or protein. More recently, a new technique involving the use of a membrane anchor sequence to effect screening for secreted protein was described in U.S. Pat. No. 5,665,590. However, such a method is applicable only for cloning genes that encode cell surface receptors or secreted proteins. Moreover, the cloning method requires elaborate procedures such as immunoaffinity column chromatography, panning, and fluorescence activated cell sorting, for the detection of the secreted products. Therefore, a need exists for alternative compositions and methods applicable for classifying and identifying the ever-growing families of genes encoding proteins located in defined subcellular locations.

An ideal reagent would be a selectable library of expression vectors that can be used in a functional assay for the classification and identification of known or novel genes based on their subcellular localization patterns, without any prior knowledge of the nature of the target genes or proteins. The present invention satisfies these needs and provides related advantages as well.

### SUMMARY OF THE INVENTION

A principal aspect of the present invention is the design of expression vectors and libraries thereof to effect isolation of genes based on the subcellular locations of the encoded proteins. Such expression vectors allow a functional selection and identification of genes comprising subcellular localization sequences, which direct the encoded proteins to specific cellular locations. The functional screening assay utilizes eukaryotic cells that are susceptible to cell transformation via the action of an oncogene.

Accordingly, the present invention provides a selectable fusion gene comprising a subcellular localization sequence fused in-frame with a defective oncogene that lacks a functional subcellular localization sequence, wherein the expression of a selectable fusion gene in a cell confers cell transformation.

In another embodiment, the present invention provides an expression vector having the following characteristics: (a) a cloning site; (b) a region encoding a defective oncogene lacking a functional subcellular localization sequence; wherein upon inserting in the cloning site a gene fragment comprising a subcellular localization sequence, in-frame with the defective oncogene, expression of the vector confers cell transformation. In one aspect, the functional subcellular localization sequence facilitates the cell transformation mediated by the oncogene. In another aspect, the functional subcellular localization sequence is required for the cell-transforming activity of the oncogene.

In a separate embodiment, the present invention provides a selectable library comprising a plurality of the above-mentioned expression vectors. In one aspect, the expression vectors contain gene fragments inserted in-frame with the defective oncogene. In another aspect, each vector contains a gene fragment that is unique with respect to all other gene fragments contained in other vectors of the same library.

In yet another separate embodiment, the present invention provides a selectable library comprising a plurality of expression vectors, wherein at least one vector has the following structural features: (a) a cloning site; (b) a region encoding a non-constitutively active oncogene; wherein upon inserting in the cloning site a gene fragment comprising a subcellular localization sequence, in-frame with the non-constitutively active oncogene, the expression thereof results in constitutive activation of the oncogene and cell transformation. The library may contain a subset of genes, or cDNAs as pooled from multiple clones or isolated from subtractive tissues.

The vectors of the present invention can contain genes or gene fragments that comprise a signal sequence(s), transmembrane anchorage domain(s) or nuclear localizaiton sequence(s). Accordingly, the inserted gene fragments may encode a secreted protein, a membrane-bound protein or a nuclear protein. In addition, the oncogenes contained in the subject vectors can be defective or non-constitutively active oncogenes. Preferred defective oncogenes are defective v-sis, ras, src, v-fos, hedgehog, Wnt1, FGF-8, FGF-9, Mob-5, WISP-1, Int2, and matrix metalloproteinase genes, which generally lack a functional subcellular localization sequence. A preferred non-constitutively active oncogene is c-raf. Furthermore, the vectors of the present invention may adopt various configurations having, e.g., the cloning site placed 3′ or preferably 5′ to the oncogene region. The vectors can also have multiple cloning sites, more than one selectable marker, origin of replication, constitutive or inducible promoters, and terminator sequences. The vectors of this invention encompass both viral and non-viral vectors.

The present invention also provides host cells comprising the expression vectors and libraries thereof. The host cells can be eukaryotic cells derived from human, mouse, rat, fruit fly, Chinese hamster, or worm. Preferred host cells are mammalian cells that can be transformed by the selected oncogenes.

The present invention further provides a method for conferring a transformation phenotype on a eukaryotic cell by introducing into the cell a subject expression vector.

Also embodied in the invention is a method of isolating a gene fragment comprising a functional subcellular localization sequence. The method involves: (a) transfecting a population of non-transformed cells a subject library of expression vectors; (b) culturing the transfected cells; (c) identifying transformed cells; and (d) isolating the gene fragment comprising the functional subcellular localization sequence from the cells exhibiting a transformation phenotype.

Also included in the invention is a method of determining subcellular location of a polypeptide. The method comprises the following steps: (a) providing an expression vector having a polynucleotide encoding the polypeptide, wherein the polynucleotide is fused in-frame with a defective oncogene or a non-constitutively active oncogene, and wherein the subcellular location at which the oncoprotein encoded by the oncogene acts to transform a cell is known; (b) transfecting a population of non-transformed cells with the expression vector; and (c) culturing the transfected cells under conditions and for a time sufficient for expression of the oncogene and sufficient for cells to exhibit a transformation phenotype, wherein an observation of cell transformation indicates that the polypeptide is located in the subcellular location where the oncoprotein acts to transform the cell.

Finally, the present invention provides kits comprising the expression vectors or libraries thereof in suitable packaging.

### BRIEF DESCRIPTION OF THE DRAWINGS

FIG. 1 is a schematic representation depicting the interaction between the oncogene v-sis and the platelet-derived growth factor receptor.

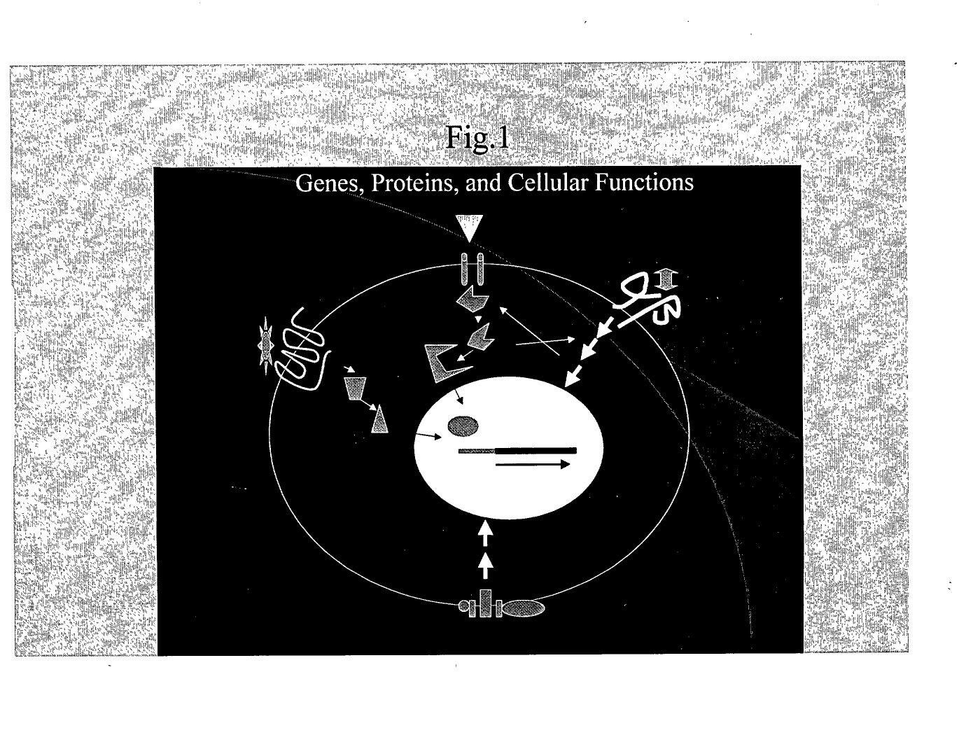

FIG. 2 depicts a simplified structure of an exemplary vector that contains a defective v-sis oncogene lacking the signal sequence. The vector is suited for isolating genes comprising a signal sequence.

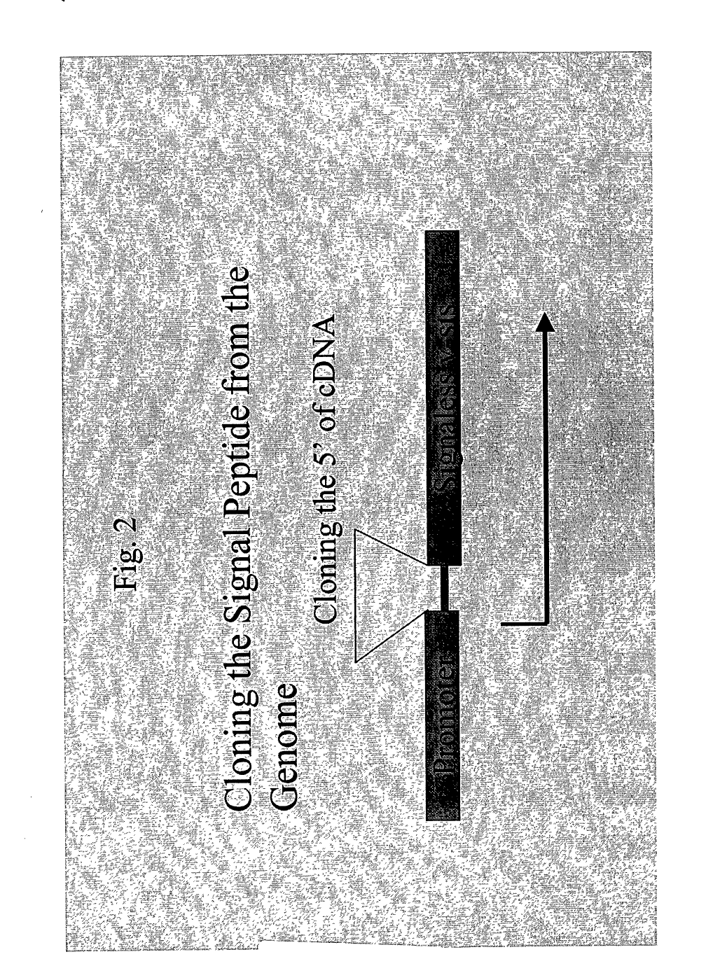

FIG. 3 depicts a simplified structure of an exemplary vector that contains a non-constitutively active c-raf The vector is applicable for isolating genes comprising a membrane anchorage domain, specifically a transmembrane domain (TM).

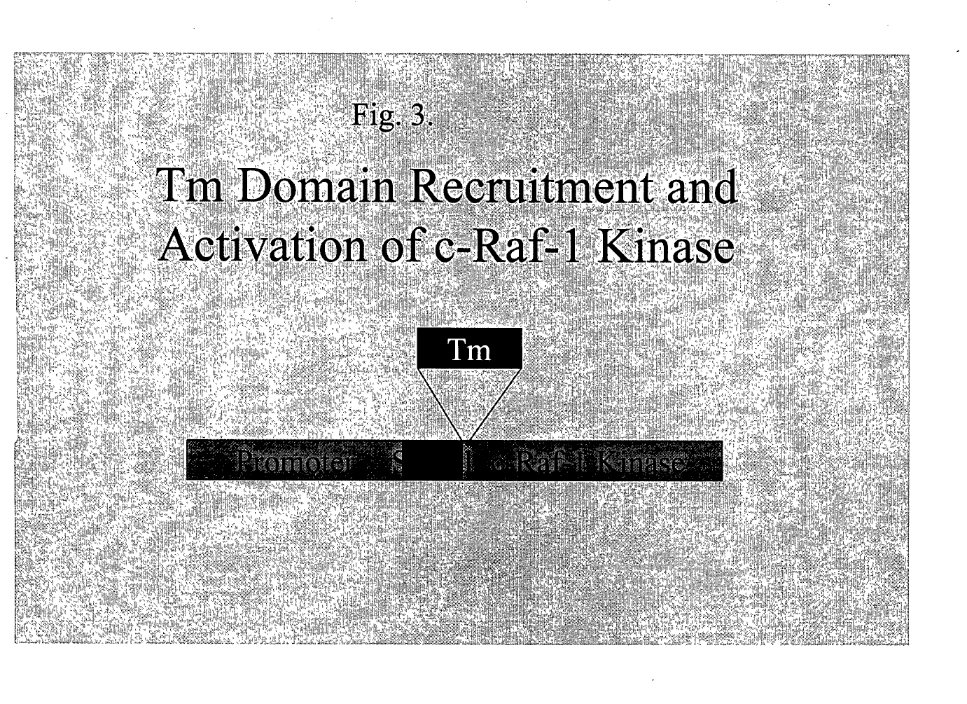

FIG. 4A depicts a simplified structure of an exemplary vector which contains a Tac antigen sequence fused in-frame with a signal sequence. This construct is incapable of transforming NIH 3T3 cells for lacking an oncogenic sequence. FIG. 4B depicts a simplified structure of an exemplary vector which contains a c-raf-1 sequence. This construct also is incapable of transforming NIH 3T3 cells because the c-raf-1 sequence is non-constitutively active. FIG. 4C depicts a simplified structure of an exemplary vector which contains the c-raf-1 sequence fused in-frame with the Tac antigen sequence and the signal sequence. Upon transfecting the NIH 3T3 cells with the vector depicted in 4C, the cells are expected to exhibit a transforming phenotype. Thus, this vector is applicable for isolating genes comprising a membrane anchorage domain, specifically a transmembrane domain (TM).

### MODE(S) FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

General Techniques

The practice of the present invention will employ, unless otherwise indicated, conventional techniques of immunology, biochemistry, chemistry, molecular biology, microbiology, cell biology, genomics and recombinant DNA, which are within the skill of the art. See, e.g., Matthews, PLANT VIROLOGY, 3^{rd} edition (1991); Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

As used in the specification and claims, the singular form “a”, “an” and “the” include plural references unless the context clearly dictates otherwise. For example, the term “a cell” includes a plurality of cells, including mixtures thereof.

Definitions

The terms “polypeptide”, “peptide” and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “membrane proteins” or “membrane-bound” or “membrane-associated proteins” are used interchangeably to refer to proteins that are directly associated with a cellular membrane structure. The terms include peripheral and integral membrane polypeptides, as well as modified cytosolic proteins that are bound directly (e.g. via a fatty acid chain) to any cellular membranes including plasma membranes and membranes of intracellular organelles.

“Cell surface receptors” represent a subset of membrane proteins, capable of binding to their respective ligands. Cell surface receptors are molecules anchored on or inserted into the cell plasma membrane. They constitute a large family of proteins, glycoproteins, polysaccharides and lipids, which serve not only as structural constituents of the plasma membrane, but also as regulatory elements governing a variety of biological functions.

The terms “membrane”, “cytosolic”, “nuclear” and “secreted” as applied to cellular proteins specify the extracellular and/or subcellular location in which the cellular protein is mostly, predominantly, or preferentially localized. By “localized” is meant that the protein is associated with, preferably predominantly associated with, and even more preferably exclusively associated with a particular cellular structure, location or compartment. Certain proteins are “chaperons,” capable of translocating back and forth between the cytosol and the nucleus of a cell.

“Domain” refers to a portion of a protein that is physically or functionally distinguished from other portions of the protein or peptide. Physically-defined domains include those amino acid sequences that are exceptionally hydrophobic or hydrophilic, such as those sequences that are membrane-associated or cytoplasm-associated. Domains may also be defined by internal homologies that arise, for example, from gene duplication. Functionally-defined domains have a distinct biological function(s). The ligand-binding domain of a receptor, for example, is that domain that binds ligand. Functionally-defined domains need not be encoded by contiguous amino acid sequences. Functionally-defined domains may contain one or more physically-defined domain. Receptors, for example, are generally divided into the extracellular ligand-binding domain, a transmembrane domain, and an intracellular effector domain. A “membrane anchorage domain” refers to the portion of a protein that mediates membrane association. Generally, the membrane anchorage domain is composed of hydrophobic amino acid residues. Alternatively, the membrane anchorage domain may contain modified amino acids, e.g. amino acids that are attached to a fatty acid chain, which in turn anchors the protein to a membrane.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “gene” or “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof.

“Operably linked” or “operatively linked” refers to a juxtaposition wherein the components so described are in a relationship permitting them to function in their intended manner. For instance, a promoter sequence is operably linked to a coding sequence if the promoter sequence promotes transcription of the coding sequence.

“Heterologous” means derived from a genotypically distinct entity from the rest of the entity to which it is being compared. For example, a promoter removed from its native coding sequence and operatively linked to a coding sequence other than the native sequence is a heterologous promoter.

A “fusion gene” is a gene composed of at least two heterologous polynucleotides that are linked together.

An “oncogene” refers to a polynucletide containing at least one open reading frame that confers a cell transformation phenotype when introduced into a host cell. Oncogenes are often altered forms of the cellular counterpart, namely the “proto-oncogenes” that are incapable of cell transformation when expressed at the level present in a non-cancer cell. The protein product of an oncogene is termed “oncoprotein.”

As used herein, “cell transformation” or “transforming phenotype” refers to the neoplastic state of a cell (a set of in vitro characteristics associated with a tumorigenic ability in vivo) include a more rounded cell morphology, looser substratum attachment, loss of contact inhibition, loss of anchorage dependence, and decreased serum requirement for cell growth in vitro.

A “subcellular localization sequence” as applied to polynucleotide or polypeptide of the subject invention refers to a sequence that facilitates transporting or confining a protein to a defined subcellular location. Defined subcellular locations include extracellular space (occupied by e.g. secreted proteins), nucleus, endoplasmic reticulum (ER), Golgi apparatus, coated pits, mitochondria, endosomes, and lysosomes.

A gene “database” denotes a set of stored data which represent a collection of sequences including nucleotide and peptide sequences, which in turn represent a collection of biological reference materials.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectively referred to as gene product. If the polynucleotide is derived from genomic DNA, expression may include splicing of the mRNA in a eukaryotic cell.

A “cell line” or “cell culture” denotes bacterial, plant, insect or higher eukaryotic cells grown or maintained in vitro. The descendants of a cell may not be completely identical (either morphologically, genotypically, or phenotypically) to the parent cell.

A “subject” as used herein refers to a biological entity containing expressed genetic materials. The biological entity is preferably plant, animal, or microorganisms including bacteria, viruses, fungi, and protozoa. Tissues, cells and their progeny of a biological entity obtained in vivo or cultured in vitro are also encompassed.

A “vector” is a nucleic acid molecule, preferably self-replicating, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions.

An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

A “replicon” refers to a polynucleotide comprising an origin of replication (generally referred to as an ori sequence) which allows for replication of the polynucleotide in an appropriate host cell. Examples of replicons include episomes (such as plasmids), as well as chromosomes (such as the nuclear or mitochondrial chromosomes).

### Vectors and Selectable Libraries of the Present Invention

As noted above, discerning the subcellular localization of a protein is of prime importance in elucidating the biological functions of a protein. Accordingly, a central aspect of the present invention is the design of a selectable expression vector library useful for the classification and identification of genes or gene fragments based on the subcellular locations of the encoded proteins. The invention library of vectors is particularly suitable for cloning genes encoding membrane bound proteins, extracellular or secreted proteins.

Distinguished from the previously described expression libraries, the subject vector libraries employ altered oncogenes whose cell transforming activities are enhanced only when expressed in-frame with a desired gene fragment. The desired gene fragment provides a subcellular localization sequence that is capable of directing the fusion product to a desired subcellular location where the oncoprotein acts to transform a cell. In one aspect, the selectable library contains a plurality of expression vectors, wherein at least one vector has the following structural features: (a) a cloning site; (b) a region encoding a defective oncogene lacking a functional subcellular localization sequence; wherein upon inserting in the cloning site a gene fragment comprising a subcellular localization sequence, in-frame with the defective oncogene, expression of the vector confers cell transformation. In another aspect, the selectable library contains a plurality of vectors, at least one of which comprises: (a) a cloning site; (b) a region encoding a non-constitutively active oncogene; wherein upon inserting in the cloning site a gene fragment comprising a subcellular localization sequence, in-frame with the non-constitutively active oncogene, the expression thereof results in constitutive activation of the oncogene and cell transformation.

Several factors apply to the design of vectors having one or more of the above-mentioned characteristics. First, the selected oncogene or fragment thereof encodes a protein product that is capable of conferring cell transformation when being expressed and transported to an appropriate cellular location. Prior research has revealed a vast number of oncoproteins that mediate cell transformation at a specific extracellular or subcellular locations (see, e.g. Mineo et al. (1997) J. of Biol. Chem. 272 (16 ) 10345-10348; Lerner et al. (1995) J. of Biol. Chem. 270(45) 26802-26806; Stokoe et al. (1994) Science 264:1463-1467; Stokoe et al. (1997) The EMBO J. 16 (9); 2384-2396; Lee et al. (1992) J. of Cell Biol. 118 (5):1057-1070; Hart et al. (1994) J. of Cell Biol. 127 (6):1843-1857; MacArthur et al. (1995) Cell Growth Differ 6 (7):817-825; Xu et al. (2000) Genes and Dev. 14:585-595. The location-dependent transformation is generally controlled by a subcellular localization sequence present in the nascent and/or matured oncoprotein. The subcellular localization sequence can be (a) a signal sequence that directs secretion of the encoded protein product; (b) a membrane anchorage domain that allow attachment of the protein to the plasma membrane or other membraneous compartment of the cell; (c) a nulcear localization sequence that mediates the translocation of the encoded protein to the nucleus; (d) an endoplasmic reticulum retention sequence that confines the encoded protein primarily to the ER; or (e) any other sequences that play a role in differential subcellular distribution of a encoded protein product. Alternatively, the location-specific cell transformation depends on the interaction between a cytosolic oncoprotein with a secondary messenger(s), e.g. a membrane anchor or a chaperon protein, which recruits the oncoprotein to the proper cellular location, where activation of cell transformation takes place.

A second consideration in-designing the subject vectors is to ensure that the vector comprises a region that encodes either a non-constitutively active oncogene, or a defective oncogene. By “defective” is meant that the oncogene exhibits reduced or preferably undetectable cell transformation activity when compared to the wildtype counterpart. The loss of cell transformation activity is due to the lack of a native functional subcellular localization sequence that normally facilitates, or preferably is required for, cell transformation. By “native” is meant that the subcellular localization sequence is part of the non-defective oncogene sequence. As used herein, a “non-constitutively active oncogene” encodes a protein which does not contain a native subcellular localization sequence capable of directing the oncoprotein to the subcellular location where the oncoprotein acts to transform a cell. The activation of the oncoprotein's cell transformation activity therefore depends on the association with other protein(s) located in the required subcellular location.

A wealth of information on the structure of various subcellular localization sequences is known in the art. For instance, the signal sequences typically correspond to the first 5 to 30 amino acids present at the N-termini of virtually all nascent, secreted proteins and cell surface receptors. The signal sequence is typically cleaved from the protein upon translocation across the membrane. Additionally, the transmembrane domain that anchors a protein to the cell membrane generally comprises hydrophobic amino acid residues. The nuclear localization sequence typically comprises a stretch of basic amino acids. Other membrane-localization sequence including ER retention sequence, myristoylation, palmitation, and farnesylation sites are also well characterized (Nilsson et al. (1989) Cell 58:707-718; Mineo et al. (1997) J. of Biol. Chem. 272 (16) 10345-10348; Lee et al. (1992) J. of Cell Biol. 118 (5):1057-1070). Based on these and other studies, a skilled artisan can routinely identify and modify the subcellular localization sequences of existing oncogenes to construct the vectors of the present invention.

Where desired, a novel oncogene can be employed in constructing the subject vectors. In such situations, the identification of a candidate subcellular localization sequence in a given oncoprotein can be determined by conventional assays without undue experimentation. Additionally, computer modeling and searching technologies further facilitates detection of subcellular localization sequences based on sequence homologies of common domains appeared in related and unrelated genes. Non-limiting examples of programs that allow homology searches are Blast (http://www.ncbi.nhn.nih.gov/BLAST/), Fasta (Genetics Computing Group package, Madison, Wis.), DNA Star, MegAlign, and GeneJocky. Any sequence databases that contains DNA sequences corresponding to target oncogenes or segments thereof can be used for sequence analysis. Commonly employed databases include but are not limited to GenBank, EMBL, DDBJ, PDB, SWISS-PROT, EST, STS, GSS, and HTGS.

For construction of the subject vectors, the choice of oncogenes will generally depend on the class of genes that is to be isolated. To clone genes encoding secreted proteins, it is preferable to use oncogenes coding for secreted proteins that mediate cell transformation outside the cell. These secreted oncoproteins include but are not limited to members of the growth factor families, extracellular proteinases, and cell matrix adhesion molecules.

Growth factors are proteins secreted by one cell and act on the cell or another cell. The oncoprotein transforms cells bearing the appropriate receptor via, e.g., an autocrine stimulation of mitogenic response. A diverse variety of growth factors have been identified. They include but are not limited to the platelet derived growth factor (PDGF), epidermal growth factor (EGF), and fibroblast growth factor (FGF) families (Cross et al. (1991) Cell 64:271-280). Preferred growth factors for construction of the subject vectors are v-sis of the PDFG family, KS/HST, Wnt1 and Int 2 of the FGF family. In addition, other FGFs including but not limited to FGF-9 and FGF-8 have been shown to transform mouse BALB/c 3T3 cells and NIH 3T3 cells, respectively (see MacArthur et al (1995) Cell Growth Differ 6 (7):817-825).

Excellular matrix proteinases (MMPs) are proteolytic enzymes capable of degrading matrix components of the basement membranes and connective tissues. It is well established that these proteinases play a central role in promoting cell metastasis and turmorgenicity.

To isolate genes whose protein products are located in a subcellular compartment, it is preferable to employ oncogene encoding proteins which transform a cell by direct or indirect association with that particular subcellular location. As used herein, subcellular compartments include but are not limited to nucleus, endoplasmic reticulum (ER), Golgi apparatus, coated pits, mitochondria, endosomes, and lysosomes. The association of the employed oncoprotein with any of these subcellular compartments may be direct or indirect. Direct association is mediated by the organelle localization sequence contained in the oncoprotein. Such sequences include but are not limited to ER retention sequence (e.g. KDEL sequence) and nuclear localization sequence as discussed above.

Of particular interest is the isolation of genes encoding nuclear proteins that have been implicated in a variety of biological responses. The subject vectors will generally employ oncogenes coding for a nuclear protein that is known to confer a cell transformation phenotype. Today, a vast number of the nuclear proteins has been elucidated and found to play a central role in mitogenic responses including cell transformation. Non-limiting examples of these oncogenic nuclear proteins are products of the transcription factor genes, such as c-fos, certain mutant retinoblastoma gene, c-jun, c-rel, and c-erbA. Other suitable genes for constructing expression vector libraries to classify and isolate genes encoding the nuclear proteins will be apparent to those skilled in the art, or will be readily ascertainable using routine experimentation.

For isolation of membrane bound proteins, it is preferable to employ oncogenes whose protein products transform a cell by direct or indirect association with a particular membraneous compartment of a cell. Oncogenes whose protein products are known to be directly associated with cell membranes include both “integral membrane” and “peripheral” polypeptides that are bound to cellular membranes including plasma membranes and membranes of intracellular organelles. An “integral membrane protein” is a transmembrane protein that extends across the lipid bilayer of the plasma membrane of a cell. A typical integral membrane protein consists of at least one “transmembrane domain” that generally comprises hydrophobic amino acid residues. An integral membrane protein may be linked to the phosphatidylinositols of the bilayer, or be held in the bilayer by a fatty acid chain, and thus can be released only by disrupting the lipid bilayer with detergents or organic solvants. Unlike the integral membrane proteins, “peripheral membrane proteins” are attached to the outer layer of a cellular membrane. They can be released from the membrane by relatively gentle extraction procedures, such as exposure to solutions of very high or low ionic strength or extreme pH. Oncogenes encoding integral membrane proteins encompass a large family of receptors including but not limited to those that interact with the growth factors disclosed herein, and any other transmembrane protein families published by Human Genome Sciences Inc., Celera, the Institute for Genomic Research (TIGR), and IncyteGenomics, Inc.

Apart from the integral and peripheral membrane oncoproteins, cytosolic oncoproteins attached to the cytoplasmic side of a membrane via a fatty acid chain can also be used. Exemplary fatty acid anchors include the myristic acid chain, palmitic acid chain that are added to a proteins with the N-terminal sequence GXXXX/S/T and CAAX, respectively. For instance, the src oncogene of Rous sarcoma virus encodes a tyrosine-specific protein kinase that is normally bound to membranes by covalently attached myristic acid chain. In this configuration the kinase can transform a cell into a cancer cell. If the attachment of this fatty acid is prevented by altering the N-terminal myristoylation sequence, the src is still active as a protein kianse, but it remains in the cytosol and does not transform the cell. Aside from src, a large family of oncoproteins with similar catalytic activities is known in the art. Non-limiting examples include c-Yes, c-Fgr, Lck, c-Fps, and Fyn are known in the art. Similar experiments have confirmed that many other oncoproteins including but not limited to GTP-binding proteins such as ras, must be bound to cell membranes via a farnesyl moiety covalently attached to the C-terminal cystein of the CAAX membrane localization sequence in order to transform cells (Jackson et al. (1990) Proc. Natl. Proc. U.S.A. 87:3042-3046; Kato et al. (1992) Proc. Natl. Proc. 89:6403-6407).

Membrane association of a cytosolic protein can also be achieved by binding to a membrane bound protein or protein complex. Accordingly, a cytosolic protein that transforms a cell upon interacting with a membrane bound protein can also be employed in screening for genes encoding membrane proteins. It is well known that many cytosolic oncoproteins, including but not limited to serine/threonine kinases, tyrosine kinases, phosphatidylinositol kinases, and GTP-binding proteins transform a cell upon associating with specific proteins anchored on the cell membrane. Such cytosolic oncoprotein is non-constitutively active when present in the cytosol. Upon association with a specific membrane anchor protein, the oncogenic protein is constitutively activated and hence capable of mediating cell transformation. A preferred example of non-constitutively active oncogene is c-raf. While c-raf is predominantly cytoplasmic, the transforming raf is associated with the membrane anchor ras protein. The recruitment of c-raf from the cytosole to the membrane activates the transforming activity of c-raf (Stokoe et al. (1994) Science 264:1463-1467; Mineo et al. (1997) J. of Biol. Chem. 272 (16) 10345-10348).

Where a non-constitutively active oncogene is selected, the entire coding region or a fragment thereof sufficient for mediating cell transformation is introduced into a recombinant expression vector. The vector containing the oncogene of this kind is constructed such that when a gene fragment encoding a subcellular localization sequence, is cloned into the cloning site in-frame with the oncogene, expression of the vector results in constitutive activation of the encoded oncoprotein and hence cell transformation.

When a constitutively active oncogene is chosen for construction of the subject vectors, the oncogene is made defective generally by altering its subcellular localization sequence. Sequence alterations can be achieved by any conventional techniques including protein manipulation procedures and recombinant DNA methods. In a preferred embodiment, the defective oncogene encodes a oncoprotein whose signal sequence is altered (e.g. by deleting the signal sequence) so that it can no longer be secreted. The resulting defective oncoprotein localizes predominantly inside the cell and remains largely non-transforming unless it is expressed in-frame with a polypeptide that carries a signal sequence. Suitable oncogenes for construction of this type of expression vectors include but are not limited to defective v-sis, ras, src, v-fos, hedgehog, certain Rb mutant, Wnt1, FGF-8, FGF-9, Mob-5, WISP-1, Int2, and matrix metalloproteinase genes.

Specifically, v-sis is a retroviral oncogene homologous to the β-chain of platelet-derived growth factor (PDGF). v-sis transforms a cell by interacting with the PDGF receptors on the surface of a cell (Lee et al. (1992) J. of Cell Biol. 118 (5):1057-1070; Hart et al. (1994) J. of Cell Biol. 127 (6):1843-1857). WISP-1 (Wnt-1 induced secreted protein 1) is a Wnt-1- and beta-catenin-responsive oncogene (Xu et al. (2000) Genes and Dev. 14:585-595). WISP-1 is a member of the CCN family of growth factors. It has been shown that overexpression of WISP-1 in normal rat kidney fibroblast cells (e.g. NRK-49F cells) induced morphological transformation, accelerated cell growth, and enhanced saturation density. The mob-5 gene is mapped to the ras/raf signaling pathway. Its expression is induced by oncogenic Ha-ras and Ki-ras, but not by normal ras. Overexpression of mob-5 may also transform cells or increase the potency of transformation of other oncogenes (Tan et al. (2000) J. Biol. Chem. 275: 24436-24443).

Another class of oncogenes suitable for constructing the subject vectors encode nuclear protein whose nuclear localization sequences are modified so that the encoded proteins are predominantly located outside of the nucleus. In one aspect, the nuclear oncogene is an altered c-fos lacking a nuclear localization sequence, and hence encoding a fos protein primarily located in the cytosol. In another aspect, the oncogene is certain mutant Rb. The vectors containing defective oncogene is designed such that when a gene fragment carrying a subcellular localization sequence is cloned into the cloning site in-frame with the defective oncogene, expression of the vectors results in the production of a fusion protein which confers a cell transformation phenotype in the recipient cells. Accordingly, the present invention also encompasses a selectable fusion gene comprising a subcellular localization sequence fused in-frame with a defective oncogene that lacks a functional subcellular localization domain, wherein the expression of the selectable fusion gene enhances the cell transformation activity of the defective oncogene.

Due to the degeneracy of the genetic code, there can be considerable variation in nucleotide sequences of the oncogenes suitable for construction of the expression vectors of the present invention. Sequence variants may have modified DNA or amino acid sequences, one or more substitutions, deletions, or additions, the net effect of which is to retain the desired cell transformation activity. For instance, various substitutions can be made in the coding region that either do not alter the amino acids encoded or result in conservative changes. These substitutions are encompassed by the present invention. Conservative amino acid substitutions include substitutions within the following groups: glycine, alanine; valine, isoleucine, leucine; aspatic acid, glutamic acid; asparagine, glutamine; serine, threonine; lysine, arginine; and phenylalanine, tyrosine. While conservative substitutions do effectively change one or more amino acid residues contained in the polypeptide to be produced, the substitutions are not expected to interfere with the cell transformation activity of the oncoprotein to be produced. Nucleotide substitutions that do not alter the amino acid residues encoded are useful for optimizing gene expression in different systems. Suitable substitutions are known to those of skill in the art and are made, for instance, to reflect preferred codon usage in the expression systems.

Where desired, the selected oncogene or gene fragment to be inserted in the vector cloning site may comprise heterologous sequences that facilitate detection of the expression and purification of the gene product. Examples of such sequences are known in the art and include those encoding reporter proteins such as β-galactosidase, β-lactamase, chloramphenicol acetyltransferase (CAT), luciferase, green fluorescent protein (GFP) and their derivatives. Other heterologous sequences that facilitate purification may code for epitopes such as Myc, HA (derived from influenza virus hemagglutinin), His-6, FLAG, or the Fc portion of immunoglobulin, glutathione S-transferase (GST), and maltose-binding protein (MBP).

The expression vectors of the present invention generally comprises a transcriptional or translational control sequences required for expressing the selected oncogene fused in-frame with a gene fragment within a cell and conferring a selectable phenotype. Suitable transcription or translational control sequences include but are not limited to replication origin, promoter, enhancer, repressor binding regions, transcription initiation sites, ribosome binding sites, translation initiation sites, and termination sites for transcription and translation.

As used herein, a “promoter” is a DNA region capable under certain conditions of binding RNA polymerase and initiating transcription of a coding region located downstream (in the 3′ direction) from the promoter. It can be constitutive or inducible. In general, the promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding domains responsible for the binding of RNA polymerase. Eukaryotic promoters will often, but not always, contain “TATA” boxes and “CAT” boxes.

The choice of promoters will largely depend on the host cells in which the vector is introduced. For animal cells, a variety of robust promoters, both viral and non-viral promoters, are known in the art. Non-limiting representative viral promoters include CMV, the early and late promoters of SV40 virus, promoters of various types of adenoviruses (e.g. adenovirus 2) and adeno-associated viruses. It is also possible, and often desirable, to utilize promoters normally associated with a desired oncogene, provided that such control sequences are compatible with the host cell system. See Goeddel et al., Gene Expression Technology Methods in Enzymology Volume 185, Academic Press, San Diego, (1991), Ausubel et al, Protocols in Molecular Biology, Wiley Interscience (1994).

Suitable promoter sequences for other eukaryotic cells include the promoters for 3-phosphoglycerate kinase, or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other promoters, which have the additional advantage of transcription controlled by growth conditions, are the promoter regions for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

In certain preferred embodiments, the vectors of the present invention use strong enhancer and promoter expression cassettes. Examples of such expression cassettes include the human cytomegalovirus immediately early (HCMV-IE) promoter (Boshart et al, Cell 41: 521,(1985)), the β-actin promoter (Gunning et al. (1987) Proc. Natl. Acad. Sci.(U.S.A) 84: 5831), the histone H4 promoter (Guild et al.(1988), J. Viral. 62: 3795), the mouse metallothionein promoter (Mclvor et al. (1987), Mol, Cell. Biol. 7: 838), the rat growth hormone promoter (Millet et al. (1985), Mol. Cell Biol. 5: 431), the human adenosine deaminase promoter (Hantzapoulos et al. (1989) Proc. Natl. Acad. Sci. U.S.A. 86: 3519), the HSV tk promoter 25 (Tabin et al. (1982) Mol. Cell. Biol. 2: 426), the α-1 antitrypsin enhancer (Peng et al. (1988) Proc. Natl. Acad. Sci. U.S.A. 85: 8146), and the immunoglobulin enhancer/promoter (Blankenstein et al. (1988) Nucleic Acid Res. 16: 10939), the SV40 early or late promoters, the Adenovirus 2 major late promoter, or other viral promoters derived from polyoma viris, bovine papilloma virus, or other retroviruses or adenoviruses. The promoter and enhancer elements of immunoglobulin (Ig) genes confer marked specificity to B lymphocytes (Baneji et al. (1983) Cell 33: 729; Gillies et al. (1983) Cell 33: 717; Mason et al. (1985) Cell 41: 479), while the elements controlling transcription of the B-globin gene function only in erythroid cells (van Assendelft et al. (1989) Cell 56:969).

Cell-specific or tissue-specific promoters may also be used. A vast diversity of tissue specific promoters have been described and employed by artisans in the field. Exemplary promoters operative in selective animal cells include hepatocyte-specific promoters and cardiac muscle specific promoters. Depending on the choice of the recipient cell types, those skilled in the art will know of other suitable cell-specific or tissue-specific promoters applicable for the construction of the expression vectors of the present invention.

Using well-known restriction and ligation techniques, appropriate transcriptional control sequences can be excised from various DNA sources and integrated in operative relationship with the intact selectable fusion genes to be expressed in accordance with the present invention.

In constructing the subject vectors, the termination sequences associated with the transgene are also inserted into the 3′ end of the sequence desired to be transcribed to provide polyadenylation of the mRNA and/or transcriptional termination signal. The terminator sequence preferably contains one or more transcriptional termination sequences (such as polyadenylation sequences) and may also be lengthened by the inclusion of additional DNA sequence so as to further disrupt transcriptional read-through. Preferred terminator sequences (or termination sites) of the present invention have a gene that is followed by a transcription termination sequence, either its own termination sequence or a heterologous termination sequence. Examples of such termination sequences include stop codons coupled to various polyadenylation sequences that are known in the art, widely available, and exemplified below. Where the terminator comprises a gene, it can be advantageous to use a gene which encodes a detectable or selectable marker; thereby providing a means by which the presence and/or absence of the terminator sequence (and therefore the corresponding inactivation and/or activation of the transcription unit) can be detected and/or selected. Alternatively, a terminator may simply be a second promoter, arranged in inverted orientation to the promoter described above.

In addition to the above-described elements, the vectors may contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will survive and/or grow under selective conditions. Typical selection genes encode protein(s) that (a) confer resistance to antibiotics or other toxins, e.g., ampicillin, neomycyin, G418, methotrexate, etc.; (b) complement auxotrophic deficiencies; or (c) supply critical nutrients not available from complex media. The choice of the proper marker gene will depend on the host cell, and appropriate genes for different hosts are known in the art.

In a preferred embodiment, the expression vector is a shuttle vector, capable of replicating in at least two unrelated expression systems. In order to facilitate such replication, the vector generally contains at least two origins of replication, one effective in each expression system. Typically, shuttle vectors are capable of replicating in a eukaryotic expression system and a prokaryotic expression system. This enables detection of protein expression in the eukaryotic host (the expression cell type) and amplification of the vector in the prokaryotic host (the amplification cell type). Preferably, one origin of replication is derived from SV40 and one is derived from pBR322 although any suitable origin known in the art may be used provided it directs replication of the vector. Where the vector is a shuttle vector, the vector preferably contains at least two selectable markers, one for the expression cell type and one for the amplification cell type. Any selectable marker known in the art or those described herein may be used provided it functions in the expression system being utilized.

The cloning site contained in the subject vector is preferably a multicloning site to allow for cloning gene fragments in all three reading frames. Any multicloning site can be used, including many that are commercially available. To facilitate expression of the gene fragment cloned into the multicloning site, the site may also include an excisable stop codon to limit background expression. In one aspect, the cloning site is placed 5′ relative to the region encoding either a defective or a non-constitutively active oncogene. Alternatively, the cloning site is arranged to the 3′ end of a defective or a non-constitutively active oncogene.

The gene or gene fragment to be inserted into the cloning site can synthetic or natural DNA molecules including genomic, or more preferably cDNA molecules. The cDNA can be synthesized by any method known in the art; preferably it is randomly primed with primers that are linked to restriction endonuclease sites found in the vector. Random priming is preferred to poly d(T) priming as it has a greater probability of obtaining the 5′ ends of genes which encode signal peptides. The cDNA fragments thus obtained are cloned into the vector which is then transfected into the expression host cell. Preferred gene fragments may be obtained from a subtracted cDNA library that is enriched with genes differentially expressed (i.e. over-expressed or under-represented) in test cells as compared to control cells. Where the test cells are tumor cells and the control cells are normal cells, the resulting subtracted cDNA library is enriched with genes that are involved in tumorigemsis.

The vectors embodied in this invention can be broadly classified into two categories: viral vectors and non-viral vectors. The latter category encompasses plasmids, cosmids, and the like. The former category includes all forms of vectors comprising sequences derived from a viral genome. Non-limiting examples are the RNA viruses such as retrovirus, and the DNA viruses such as adenovirus, adeno-associated viruses, and the like. Preferred viral vectors contain viral backbone sequences that have a minimal propensity to transform a cell.

Retroviruses carry their genetic information in the form of RNA; however, once the virus infects a cell, the RNA is reverse-transcribed into the DNA form which integrates into the genomic DNA of the infected cell. The integrated DNA form is called a provirus. Methods for constructing retroviral vectors are well established in the art and hence are not detailed herein (see, e.g., WO 92/08796).

Likewise, procedures and techniques suitable for constructing DNA viral vectors are readily available. For instance, the genomic structures of both adenovirus (Ad) or adeno-associated virus (AAV) are well characterized. Adenoviruses (Ads) represent a homogenous group of viruses, including over 50 serotypes. (see, e.g., WO 95/27071). Ads are easy to grow and do not require integration into the host cell genome. Recombinant Ad-derived vectors, particularly those that reduce the potential for recombination and generation of wild-type virus, have also been constructed (see, WO 95/00655; WO 95/11984). Wild-type AAV has high infectivity and specificity integrating into the host cells genome. (Hermonat and Muzyczka (1984) PNAS USA 81:6466-6470; Lebkowski et al. (1988) Mol. Cell. Biol. 8:3988-3996).

In general, the vectors having one or more of the above-mentioned characteristics can be obtained using recombinant cloning methods and/or by chemical synthesis. A vast number of recombinant cloning techniques such as PCR, restriction endonuclease digestion and ligation are well known in the art, and need not be described in detail herein. One of skill in the art can also use the sequence data provided herein or that in the public or proprietary databases to obtain a desired vector by any synthetic means available in the art.

### Host Cells of the Present Invention

The invention provides host cells transfected with the expression vectors or a library of the expression vectors described above. The expression vectors can be introduced into a suitable eukaryotic cell by any of a number of appropriate means, including electroporation, microprojectile bombardment; lipofection, infection (where the vector is coupled to an infectious agent), transfection employing calcium chloride, rubidium chloride, calcium phosphate, DEAE-dextran, or other substances. The choice of the means for introducing vectors will often depend on features of the host cell.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention. Preferred cells of the invention are animal cells, preferably mammalian cells, and even more preferably mammalian cells capable of being transformed in vitro via the actions of the oncogene selected for construction of the subject vectors. Examples of mammalian host cells include but not limited to NIH3T3 cells, COS, HeLa, and CHO cells.

Once introduced into a suitable host cell, expression of the gene fragment as part of the fusion oncoprotein can be determined using any assay known in the art. For example, the presence of transcribed mRNA of the fusion oncogene can be detected and/or quantified by conventional hybridization assays (e.g. Northern blot analysis), amplification procedures (e.g. RT-PCR), SAGE (U.S. Pat. No. 5,695,937), and array-based technologies (see e.g. U.S. Pat. Nos. 5,405,783, 5,412,087 and 5,445,934), using probes complementary to the oncogene or fragment thereof.

Expression of the fusion gene can also be determined by examining the oncoprotein expressed as a fusion product. A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunoflourescent assays, and PAGE-SDS.

In general, antibodies that specifically recognize and bind to the oncoprotein portion of the fusion product are required for conducting the aforementioned protein analyses. The term “antibodies” or as used herein refers to immunoglobulin molecules and antigen-binding portions of immunoglobulin molecules, i.e., molecules that contain an antigen binding site which specifically binds (“immunoreacts with”) an antigen. Structurally, the simplest naturally occurring antibody (e.g., IgG) comprises four polypeptide chains, two heavy (H) chains and two light (L) chains inter-connected by disulfide bonds. The natural immunoglobulins represent a large family of molecules that include several types of molecules, such as IgD, IgG, IgA, IgM and IgE. The term also encompasses hybrid antibodies, or altered antibodies, and fragments thereof, including but not limited to Fab fragment(s), and Fv fragment. It has been shown that the antigen-binding function of an antibody can be performed by fragments of a naturally-occurring antibody. These fragments are also termed antigen-binding fragments. Examples of binding fragments encompassed within the term antigen-binding fragments include but are not limited to (i) an Fab fragment consisting of the VL, VH, CL and CH1 domains; (ii) an Fd fragment consisting of the VH and CHI domains; (iii) an Fv fragment consisting of the VL and VH domains of a single arm of an antibody, (iv) a dAb fragment (Ward et al., (1989) Nature 341:544-546) which consists of a VH domain; (v) an isolated complimentarily determining region (CDR); and (vi) an F(ab')2 fragment, a bivalent fragment comprising two Fab fragments linked by a disulfide bridge at the hinge region. Furthermore, although the two domains of the Fv fragment are generally coded for by separate genes, a synthetic linker can be made that enables them to be made as a single protein chain (known as single chain Fv (scFv); Bird et al. (1988) Science 242:423-426; and Huston et al. (1988) PNAS 85:5879-5883) by recombinant methods. Such single chain antibodies are also encompassed within the term “antigen-binding fragments”. Preferred antibody fragments are those which are capable of crosslinking their target antigen, e.g., bivalent fragments such as F(ab')₂ fragments. Alternatively, an antibody fragment which does not itself crosslink its target antigen (e.g., a Fab fragment) can be used in conjunction with a secondary antibody which serves to crosslink the antibody fragment, thereby crosslinking the target antigen.

These antibodies may be purchased from commercial vendors or generated and screened using methods well known in the art. See Harlow and Lane (1988) supra. and Sambrook et al. (1989) supra.

The host cells of this invention can be used, inter alia, as repositories of the subject vectors, or as vehicles for screening desired genes based on the extracellular or subcellular distribution of the encoded products.

### Uses of the Vectors or the Selectable Libraries of the Present Invention

The subject vectors and libraries provide specific reagents for cloning genes or gene fragments that encode protein products expected to be preferentially localized to certain extracellular or subcellular locations. The gene cloning technique may be used in a wide variety of circumstances including classification of existing or more preferably novel genes based on the subcellular distribution patterns of their protein products; detecting protein-protein interaction by analyzing a phenotypic change in the host cell; and facilitating the elucidation of the biological functions of a variety of genes.

Accordingly, this invention provides a method of isolating a gene fragment comprising a functional subcellular localization sequence. The method comprises the steps of: a method of isolating a gene fragment comprising a functional subcellular localization sequence, the method comprising: (a) transfecting a population of non-transformed cells the selectable library of expression vectors; (b) culturing the transfected cells; (c) identifying transformed cells; and (d) isolating the gene fragment comprising the functional subcellular localization sequence from the cells exhibiting a transformation phenotype. Preferably, the transfected cells are cultured under conditions and for a time sufficient for expression of the oncogene contained in the vectors, and for cells to exhibit a transformation phenotype.

In a separate embodiment, the present invention provides a method of determining subcellular location of a polypeptide. The method involves the steps of: (a) providing an expression vector having a polynucleotide encoding the polypeptide, wherein the polynucleotide is fused in-frame with a defective oncogene or a non-constitutively active oncogene, and wherein the subcellular location at which the oncoprotein encoded by the oncogene acts to transform a cell is known; (b) transfecting a population of non-transformed cells with the expression vector; and (c) culturing the transfected cells under conditions and for a time sufficient for expression of the oncogene and sufficient for cells to exhibit a transformation phenotype, wherein an observation of cell transformation indicates that the polypeptide is located in the subcellular location where the oncoprotein acts to transform the cell.

The host cells encompassed by these embodiments are generally eukaryotic cells susceptible to transformation via the action of an oncogene. Thus, the choice of cells for the subject cloning method will depend on the type of oncogene utilized in the selectable library. Generally, suitable cells are eukaryotic cells equipped with an array of signaling molecules that is capable of transmitting the stimulatory signals triggered by a given oncogene. The transduction of the stimulatory signals may culminate in a wide range of mitogenic responses including cell transformation, which can be readily detected. Over the past decades, the signaling transduction pathways of numerous oncogenes have been delineated. A classic signaling cascade involves growth factors that stimulate cell transformation by interacting with their corresponding cell surface receptors. Upon binding to the respective growth factor receptors, the growth factor/receptor complex modifies key regulatory proteins in the cytoplasm, which in turn signal other down-stream secondary messengers to initiate cell transformation. An illustrative component of this classic signal transduction complex is the oncogenic growth factor v-sis that only transforms cells expressing the respective receptor, namely the platelet-derived growth factor (PDGF) receptor. Thus, if v-sis oncogene is used for the subject cloning methods, cells expressing the PDGF receptors should be employed. Such cells include common cell lines such as NIH 3T3 cells, BALB/ 3T3, various kinds of fibroblasts that contain endogenous PDGF receptors, or any other cells that carry exogenously introduced PDGF receptors.

As noted above, the selectable library of expression vectors is introduced into non-transformed cells to assay for the transforming phenotype caused by the desired gene or gene fragment. “Non-transformed cells” refer to cells that do not exhibit detectable transforming phenotype. Commonly observed non-transforming phenotypes of cells include but are not limited to the requirement of serum in cell culture medium, dependence on substratum for in vitro growth, and inhibition by cell-cell contract. A preferred criterion for selecting non-transformed cells is based on their inability to grow in soft agar. As is apparent to artisans in the field, many other criteria including the presence of certain tumor suppressor gene(s) (e.g. p53), the absence of dominant oncogenes can also be employed to ascertain the non-transforming phenotype of a cell.

Suitable non-transformed cells may be derived from primary cultures or subcultures generated by expansion and/or cloning of primary cultures. Any non-transformed cells capable of growth in culture can be used as host cells. The host cells may have a species origin of human, mouse, rat, fruit fly, Chinese hamster, or worm. As is known to one skilled in the art, various cell lines may be obtained from public or private repositories. The largest depository agent is American Type Culture Collection (http://www.atcc.org), which offers a diverse collection of well-characterized cell lines derived from a vast number of organisms and tissue samples.

Upon delivery of the subject library of expression vectors, the host cells are typically cultured under conditions favorable for gene transcription and/or selection for the transfected cells. The parameters governing eukaryotic cell survival are generally applicable for induction of gene transcription. The culture conditions are well established in the art. Physicochemical parameters which may be controlled in vitro are, e.g., pH, CO₂, temperature, and osmolarity. The nutritional requirements of cells are usually provided in standard media formulations developed to provide an optimal environment. Nutrients can be divided into several categories: amino acids and their derivatives, carbohydrates, sugars, fatty acids, complex lipids, nucleic acid derivatives and vitamins. Apart from nutrients for maintaining cell metabolism, most cells also require one or more hormones from at least one of the following groups: steroids, prostaglandins, growth factors, pituitary hormones, and peptide hormones to survive or proliferate (Sato, G.H., et al. in “Growth of Cells in Hormonally Defined Media”, Cold Spring Harbor Press, N.Y., 1982; Ham and Wallace (1979) Meth. Enz., 58:44, Barnes and Sato (1980) Anal. Biochem., 102:255. Given the vast wealth of information on the nutrient requirements, medium conditions optimized for cell survival, one skilled in the art can readily fashion various culture conditions using any one of the aforementioned methods and compositions, alone or in any combination.

In general, the transfected cells are also cultured for a sufficient amount of time for the development of a transforming phenotype. The amount of time required will vary depending on the transformation assay that is employed for the study. Generally, foci formation assay requires approximately 3 to 30 days, preferably 3 to 20 days, more preferably 3 to 15 days, and even more preferably 3 to 10 days. For soft agar assay, approximately the same period of time is required to observe growth of the transfected cells. The detailed experimental procedures and variations thereof for carrying out these and other cell transformation assays are well established in the art, and thus are not further detailed herein.

In assaying for cell transformation, one typically conducts a comparative analysis of test cells and appropriate control cells. Preferably, the analysis includes positive control cells exhibiting transforming phenotype upon transfection and expression of a constitutively active oncogene. More preferably, the analysis includes negative control cells that are transfected with control vectors carrying only a defective oncogene, or a non-constitutively active oncogene, or no oncogenic sequences at all.

The cells transformed by an expression vector provide specific reagents for isolating and cloning the target genes or gene fragments that comprise functional subcellular localization sequences. The subcellular localization sequences typically direct the encoded protein to the respective subcellular locations. As used herein, the term “isolated” means separated from constituents, cellular and otherwise, in which the gene or fragments thereof, are normally associated with in nature.

The genes or gene fragments contained in the transformed cells can be isolated by a number of processes well known to artisans in the field. A representative procedure is expression cloning by immunoprecipitation and immunoaffinity purification of the target protein as a fusion of the oncoprotein encoded by the expression vectors from cell lysates. Both methods proceed with binding the target fusion protein to antibodies (specific for the oncoprotein portion or a tag sequence) that are immobilized onto a solid-phase matrix (e.g. protein A and protein G sepharose beads), followed by separating the bound antigens with the unbound proteins, and finally eluting the antigens from the antibody-coupled solid-phase matrix. Subsequent analysis of the eluted fusion may involve electrophoresis for determining the molecular weight, and protein sequencing for delineating the amino acid sequences of the target antigen. Based on the deduced amino acid sequences, the cDNA encoding the desired gene or gene fragment can then be obtained by recombinant cloning methods including PCR, library screening, homology searches in existing nucleic acid databases, or any combination thereof. Commonly employed databases include but are not limited to GenBank, SWISSPROT, EST, HTGS, GSS, EMBL, DDBJ, PDB and STS.

A preferred method of cloning the target gene or gene fragments is to obtain the cDNAs of the transformed cells. cDNAs can be obtained by reverse transcribing the mRNAs from a particular cell type according to standard methods in the art. Specifically, mRNA can be isolated using various lytic enzymes or chemical solutions according to the procedures set forth in Sambrook et al. (“Molecular Cloning: A Laboratory Manual”, Second Edition, 1989), or extracted by nucleic-acid-binding resins following the accompanying instructions provided by manufacturers. The nucleotide sequence of the synthesized cDNAs can then be determined by direct sequencing using an automated sequencer. Alternatively, the cDNA can be sequenced by hybridization assays, amplification procedures (e.g. PCR, SAGE (U.S. Pat. No. 5,695,937), and array-based technologies (see e.g. U.S. Pat. Nos. 5,405,783, 5,412,087 and 5,445,934).

The genes or gene fragments identified by the subject cloning methods are non-ubiquitously expressed genes, whose protein products exhibit a restricted subcellular expression patterns. In one aspect, the gene or fragment comprises a functional signal sequence and encodes a secreted polypeptide. In another aspect, the gene or fragment contains a functional membrane anchorage domain (e.g. transmembrane domain, myristoylation or palmitation sequence) and encodes a membrane polypeptide. In yet another aspect, the gene or fragment carries a nuclear localization sequence that directs the encoded protein to the nucleus. In still yet another aspect, the isolated gene contains an ER retention sequence that confines the encoded protein to the ER region.

The isolated genes or gene fragments of the present invention may further be characterized based on one or more of the following features: ability to induce a phenotypic change in a host cell or organism, species origin, developmental origin, primary structural similarity, involvement in a particular biological process, association with or resistance to a particular disease or disease stage. In one aspect, the isolated gene may be any eukaryotic gene expressed in a eukaryote cell, such as a plant cell, animal cell or a yeast cell. In another aspect, the isolated gene confers a phenotypic characteristic detectable by visual, microscopic, genetic, or chemical means. Within this class of genes, of particular interest are genes involved in cell growth control.

In another aspect, the isolated genes are of a specific developmental origin, such as those expressed in an embryo or an adult organism, during ectoderm, mesoderm, or endoderm formation in a multi-cellular animal. In yet another aspect, the isolated genes are involved in a specific biological process, including but not limited to cell cycle regulation, cell differentiation, chemotaxsis, apoptosis, cell motility and cytoskeletal rearrangement. In still another aspect, the isolated endogenous genes embodied in the invention are associated with a particular disease or with a specific disease stage. Such genes include but are not limited to those associated with obesity, hypertension, diabetes, autoimmune diseases, neuronal and/or muscular degenerative diseases, cardiac diseases, endocrine disorders, any combinations thereof.

### Kits Comprising the Vectors or Selectable Libraries of the Present Invention

The present invention also encompasses kits containing the vectors or libraries of vectors of this invention in suitable packaging. Kits embodied by this invention include those that allow isolation of genes or gene fragments comprising functional subcellular localization sequences. The encoded proteins are expected to be predominantly located in certain subcellular or extracellular compartments.

Each kit necessarily comprises the reagents which render the delivery of vectors into a eukaryotic host cell possible. The selection of reagents that facilitate delivery of the vectors may vary depending on the particular transfection or infection method used. The kits may also contain reagents useful for generating labeled polynucleotide probes or proteinaceous probes for detection of gene or protein expression. Each reagent can be supplied in a solid form or dissolved/suspended in a liquid buffer suitable for inventory storage, and later for exchange or addition into the reaction medium when the experiment is performed. Suitable packaging is provided. The kit can optionally provide additional components that are useful in the procedure. These optional components include, but are not limited to, buffers, capture reagents, developing reagents, labels, reacting surfaces, means for detection, control samples, instructions, and interpretive information. The kits can be employed to classify and/or identify genes encoding proteins localized to defined extracellular/subcellular locations.

Further illustration of the development and use of vectors and assays according to this invention are provided in the Example section below. The examples are provided as a guide to a practitioner of ordinary skill in the art, and are not meant to be limiting in any way.

### Example 1

### Construction of Selectable Library of Expression Vectors Using a Defective Oncogene-Signal Peptide (SP) Mediates v-sis Protein Secretion and Transforming Activity

Oncogenic transformation of NIH3T3 or Rat-1 cells by v-sis requires the protein to be secreted and interacts with the cognate receptor. The v-sis contains signal peptide at its N-terminal, followed by a propeptide with a dibasic proteolytic processing site, and the 82-amino acid minimal transforming regions. To use v-sis transforming activity as an indicator or reporter for signal peptide, the signal peptide of v-sis is deleted, and cloned into a vector pcDNA3, under the control of pCMV promoter. Multiple cloning sites are placed between the promoter and the v-sis transforming gene. A library of selected gene fragments, or certain specific gene fragment is cloned into the multiple cloning sites, and the library is amplified in E. coli. Briefly, the resulting library is transfected into NIH3T3 cells or Rat-1 cells, and soft agar growth and/or focus formation are scored, both of which are indicative of cell transformation, demonstrating that a gene or gene fragment encoding a signal peptide is cloned upstream of the v-sis protein, leading to the secretion of the v-sis protein. The colonies in the soft agar are isolated and the cells are expanded. DNA is isolated from those cells, and the insert coding for the signal sequence is amplified by PCR, using primer pairs, one of which corresponding to the pCMV promoter region, another being complementary to part of the v-sis coding sequences. The isolated gene insert may be a full length gene or a partial sequence. Based on the partial sequence of the insert, the full length sequence is identified using conventional molecular biology techniques as described (Sambrook et al., Molecular Cloning). The activity of the identified signal peptide can be further confirmed using conventional molecular and cellular biology techniques.

### Example 2

### Construction of Selectable Library of Expression Vectors Using a Non-Constitutively Active Oncogene-Membrane Localization Sequence and/or Transmembrane Domain (Tm) Anchors c-raf-1 to the Cytoplasmic Membrane and Leads to the c-raf-1 Activation

The mechanism by which Ras transforms cell is to recruit raf to the cytoplamic membrane, where raf is activated and associated with plasma membrane cytoskeleton elements. When raf is engineered to contain the C-terminal 17 amino acids of K-ras, that contains the CAAX motif for membrane targeting, C-raf-1 becomes constitutively active (D. Stokoe et al., 1994, Science 264: 1463-1467). To use c-raf-1 transforming activity as an indicator or reporter for membrane localization sequences, the c-raf-1 is cloned into a vector pcDNA3, under the control of pCMV promoter. Multiple cloning sites are placed between the promoter and the c-raf-1 proto-oncogene. A library of selected gene fragment, or certain specific gene fragment is cloned into the multiple cloning sites, and the library is amplified in E.-Coli. Briefly, the library is transfected into NIH3T3 cells or Rat-1 cells, and soft agar growth and/or focus formation are scored, both of which are indicative of oncogenic activity, demonstrating that a gene or gene fragment encoding a membrane localization sequence or transmembrane domain is cloned upstream of the raf-1 protein, leading to the membrane localization and activation of c-raf-1 protein. The colonies in the soft agar are isolated and the cells are expanded. DNA is isolated from those cells, and the insert coding for the membrane localization sequence or transmembrane domain is amplified by PCR, using primer pairs, one of which corresponds to the pCMV promoter region, another of which is complementary to part of the c-raf-1 coding sequences. The isolated gene insert may be a full-length gene or a partial sequence. Based on the partial sequence of the insert, the full length of the gene is identified using conventional molecular biology techniques as described (Sambrook et al., Molecular Cloning). The activity of the identified signal peptide can be further confirmed using conventional molecular and cellular biology techniques.

### Example 3

### Constructs Expressing Transmembrane Domain (Tm) from CD25 (Also Called Tac Antigen) Anchor c-raf-1 to the Cytoplasmic Membrane and Lead to the c-raf-1 Activation and Cellular Transformation

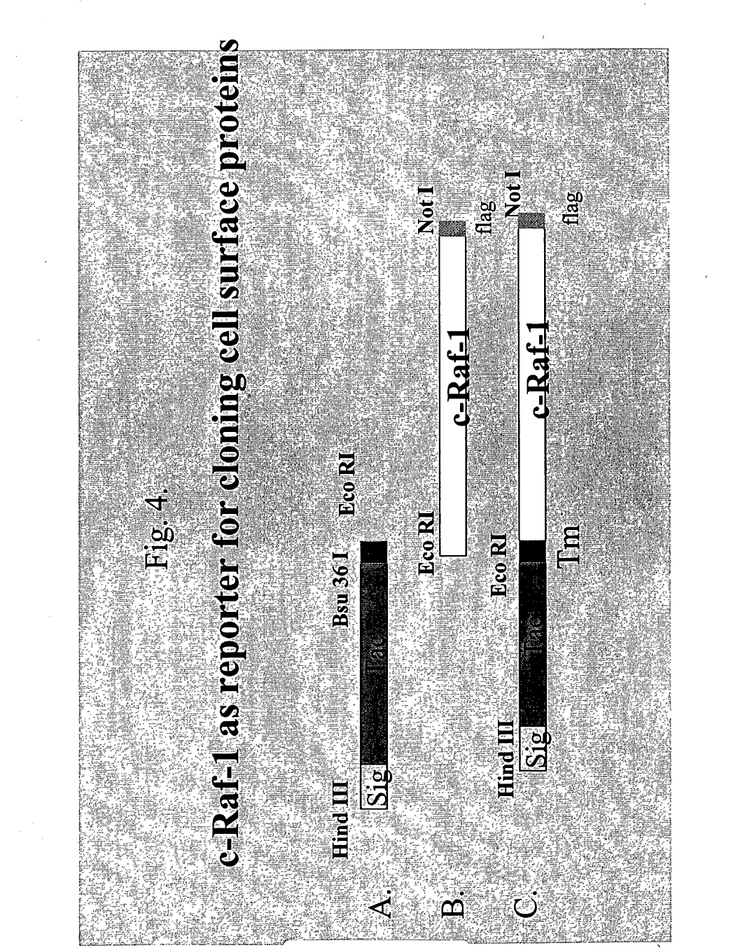

The CD25 (Tac antigen) is the alpha subunit of interleukin 2 receptor (IL-2R) that contains a short cytoplasmic tail. The cDNA encoding the CD25 is amplified from a cDNA library. Upon linking the Hind III and Eco RI cloning sites, the IL-2R fragment is cloned into the pSF80 vector (FIG. 4A) using conventional molecular biology techniques (e.g. as described in Sambrook et al., Molecular Cloning). The expression of the Tac antigen alone (FIG. 4A) does not bind to the ligand interleukin 2 (IL-2) and is expected to be incapable of transforming cells such as NIH3T3 or Rat-1 cells.

Another pSF80 construct (FIG. 4B) containing c-raf-1 (Li et al., (1995) EMBO J. 14(4):685) is constructed. The c-raf-1 sequence is placed under the control of pCMV promoter. As indicated above, full-length c-raf-1 in and by itself does not transform cells. By contrast, a construct containing the full-length c-raf-1 gene fused in-frame with the Tac antigen with the signal peptide (FIG. 4C), is expected to transform NIH3T3 or Rat-1 cells. In this case, the c-raf-1 protein is brought to the cytoplasmic membrane via the signal peptide of the Tac antigen. Upon associating with the membrane, c-raf-1 is activated, and thereby transforming the cells as evidenced by foci formation or the ability of the cell to grow in soft agar. This system allows one to isolate and identify genes or fragments encoding a membrane localization sequence or transmembrane domain.

What is claimed is:

1. A selectable fusion gene comprising a subcellular localization sequence fused in-frame with a defective oncogene that lacks a functional subcellular localization sequence, wherein the selectable fusion gene when expressed in a cell confers cell transformation.

2. The selectable fusion gene of claim 1, wherein the cell transformation is characterized by a phenotypic change selected from the group consisting of formation of cell foci, reduced requirement of serum for cell growth in vitro, and loss of anchorage dependence.

3. The selectable fusion gene of claim 2, wherein the loss of anchorage dependence is further characterized by cell growth in soft agar.

4. The selectable fusion gene of claim 1, wherein the functional subcellular localization sequence is required for the cell transforming activity of the oncogene.

5. The selectable fusion gene of claim 1, wherein the subcellular localization sequence encodes a signal peptide.

6. The selectable fusion gene of claim 1, wherein the subcellular localization sequence encodes a membrane anchorage domain.

7. The selectable fusion gene of claim 1, wherein the subcellular localization sequence encodes a nuclear localization sequence.

8. The selectable fusion gene of claim 1, wherein the defective oncogene is a defective v-sis that lacks a fundamental subcellular localization sequence.

9. The selectable fusion gene of claim 1, wherein the defective oncogene is selected from the group consisting of defective ras, src, v-fos, hedgehog, Wnt1, FGF-8, FGF-9, Mob-5, WISP-1, Int2, and matrix metalloproteinase genes.

10. An expression vector, comprising:
(a) a cloning site;
(b) a region encoding a defective oncogene lacking a functional subcellular localization sequence;
wherein upon inserting in the cloning site a gene fragment comprising a subcellular localization sequence, in-frame with the defective oncogene, expression thereof confers cell transformation.

11. The expression vector of claim 10, wherein the gene fragment comprising a subcellular localization sequence is inserted in-frame with the defective oncogene, expression thereof confers cell transformation.

12. The expression vector of claim 10, wherein the functional subcellular localization sequence is required for the cell transforming activity of the oncogene.

13. The expression vector of claim 10, wherein the cloning site of (a) and the region of (b) are arranged from 5′ to 3′.

14. The expression vector of claim 10, wherein the region of (b) and the cloning site of (a) are arranged from 5′ to 3′.

15. The expression vector of claim 10, wherein the cloning site is a multiple cloning site.

16. The expression vector of claim 10, wherein at least one nucleotide is added or subtracted to the cloning site to facilitate the expression of gene fragment in multiple reading frames.

17. The expression vector of claim 15, wherein the multiple cloning site contains an excisable stop codon.

18. The expression vector of claim 10, further comprising at least two origins of replication, wherein at least one first origin facilitates replication in an expression cell type, and at least one second origin facilitates replication in an amplification cell type.

19. The expression vector of claim 10, further comprising at least one gene encoding a selectable marker.

20. The expression vector of claim 18, wherein the expression cell type is eukaryotic and the amplification cell type is prokaryotic.

21. The expression vector of claim 19, wherein the selectable marker facilitates selection in an expression cell type.

22. The expression vector of claim 19, wherein the selectable marker facilitates selection in an amplification cell type.

23. The expression vector of claim 18, wherein the origins of replication are derived from SV40 and pBR322.

24. The expression vector of claim 10, further comprising a promoter 5′ to the cloning site.

25. The expression vector of claim 24, wherein the promoter is a constitutive promoter.

26. The expression vector of claim 24, wherein the promoter is an inducible promoter.

27. The expression vector of claim 24, wherein the promoter is a tissue-specific promoter.

28. The expression vector of claim 10, further comprising a terminator immediately 3′ to the region of (b).

29. The expression vector of claim 10, wherein the vector is a viral vector selected from the group consisting of retroviral vector, adeno-associate vial vector, and adenoviral vector.

30. The expression vector of claim 10, wherein the vector is a non-viral vector.

31. The expression vector of claim 10, wherein the cell transformation is characterized by a phenotypic change selected from the group consisting of formation of cell foci, reduced requirement of serum for cell growth in vitro, and loss of anchorage dependence.

32. The expression vector of claim 31, wherein the loss of anchorage dependence is further characterized by cell growth in soft agar.

33. The expression vector of claim 10, wherein the subcellular localization sequence encodes a signal peptide.

34. The expression vector of claim 10, wherein the subcellular localization sequence encodes a transmembrane domain.

35. The expression vector of claim 10, wherein the subcellular localization sequence encodes a nuclear localization sequence.

36. The expression vector of claim 10, wherein the defective oncogene is a defective v-sis that lacks a functional subcellular localization sequence.

37. The expression vector of claim 10, wherein the defective oncogene is selected from the group consisting of a defective ras, src, v-fos, hedgehog, Wnt1, FGF-8, FGF-9, Mob-5, WISP-1, Int2, and matrix metalloproteinase genes.

38. The expression vector of claim 10, wherein the gene fragment encodes a polypeptide selected from the group consisting of a membrane bound protein, a secreted protein, and a nuclear protein.

39. The expression vector of claim 10, wherein the gene fragment encodes an animal protein or a plant protein.

40. A selectable library comprising a plurality of expression vectors, at least one being a vector of claim 10.

41. A selectable library comprising a plurality of expression vectors at least one being a vector of claim 11.

42. A selectable library comprising a plurality of expression vectors, wherein at least one vector comprises:
(a) a cloning site;
(b) a region encoding a non-constitutively active oncogene, wherein upon inserting in the cloning site a gene fragment comprising a subcellular localization sequence, in-frame with the non-constitutively active oncogene, the expression thereof results in constitutive activation of the oncogene and cell transformation.

43. The selectable library of claim 42, wherein the gene fragment is inserted in-frame with the non-constitutively active oncogene.

44. The selectable library of claim 42, wherein the non-constitutively active oncogene is c-raf.

45. A host cell comprising the expression vector of claim 10 or 11.

46. A population of host cells transfected with a selectable library of claim 41 or 43.

47. The population of host cells of claim 46, where the cells are eukaryotic cells.

48. The population of eukaryotic host cells of claim 47, where the cells have a species origin selected from the group consisting of human, mouse, rat, fruit fly, Chinese hamster, and worm.

49. A method for conferring a transformation phenotype on a eukaryotic cell, comprising the step of introducing into the cell an expression vector according to claim 11.

50. A method of isolating a gene fragment comprising a functional subcellular localization sequence, the method comprising:
(a) transfecting a population of non-transformed cells a selectable library of expression vectors of claim 41 or 43;
(b) culturing the transfected cells;
(c) identifying transformed cells; and
(d) isolating the gene fragment comprising the functional subcellular localization sequence from the cells exhibiting a transformation phenotype.

51. A method of isolating a gene fragment comprising a functional subcellular localization sequence, the method comprising:
(a) providing a selectable library of expression vectors of claim 41 or 43;
(b) transfecting a population of non-transformed cells with the library of expression vectors;
(c) culturing the transfected cells under conditions and for a time sufficient for expression of the oncogene, and sufficient for cells to exhibit a transformation phenotype; and
(d) isolating the gene fragment comprising the functional subcellular localization sequence from the cells exhibiting a transformation phenotype.

52. The method of claim 51, wherein the gene fragment encodes a polypeptide with a restricted subcellular expression pattern.

53. The method of claim 51, wherein the gene fragment encodes an animal protein or a plant protein.

54. The method of claim 51, wherein the gene fragment comprises a functional signal sequence and encodes a secreted polypeptide.

55. The method of claim 51, wherein the gene fragment comprises a functional membrane anchorage domain and encodes a membrane protein.

56. The method of claim 51, wherein the membrane anchorage domain is a transmembrane domain of an integral membrane protein.

57. The method of claim 51, wherein the gene fragment comprises a functional nuclear localization sequence, and encodes a nuclear protein.

58. The method of claim 51, where the non-transformed cells are eukaryotic cells.

59. The method of claim 51, where the non-transformed cells are mammalian cells.

60. The method of claim 51, where the non-transformed cells have a species origin being selected from the group consisting of human, mouse, rat, fruit fly, Chinese hamster, and worm.

61. The method of claim 51, wherein the gene fragment is fused in-frame from 5′ to 3′ with the oncogene.

62. The method of claim 51, wherein the gene fragment is fused in-frame from 3′ to 5′ with the oncogene.

63. The method of claim 51, wherein the vector further comprises at least two origins of replication, wherein at least one first origin facilitates replication in an expression cell type, and at least one second origin facilitates replication in an amplification cell type.

64. The method of claim 51, wherein the vector further comprises at least one gene encoding a selectable marker.

65. The method of claim 63, wherein the expression cell type is eukaryotic and the amplification cell type is prokaryotic.

66. The method of claim 64, wherein the at least one selectable marker facilitates selection in an expression cell type.

67. The method of claim 64, wherein the at least one selectable marker facilitates selection in an amplification cell type.

68. The method of claim 63, wherein the origins of replication are derived from SV40 and pBR322.

69. The method of claim 51, wherein the cell transforming is characterized by a phenotypic change selected from the group consisting of formation of cell foci, reduced requirement of serum for cell growth in vitro, and loss of anchorage dependence.

70. The method of claim 51, wherein the gene fragment comprises genomic DNA.

71. The method of claim 51, wherein the gene fragment comprises cDNA.

72. The method of claim 51, wherein the defective oncogene is a defective v-sis.

73. The method of claim 51, wherein the defective oncogene is selected from the group consisting of a defective ras, src, v-fos, hedgehog, Wnt1, FGF-8, FGF-9, Mob-5, WISP-1, Int2, and matrix metalloproteinase genes.

74. The method of claim 51, wherein the non-constitutively active oncogene is c-raf

75. A method of determining subcellular location of a polypeptide, comprising:
(a) providing an expression vector having a polynucleotide encoding the polypeptide, wherein the polynucleotide is fused in-frame with a defective oncogene or a non-constitutively active oncogene, and wherein the subcellular location at which the oncoprotein encoded by the oncogene acts to transform a cell is known;
(b) transfecting a population of non-transformed cells with the expression vector; and
(c) culturing the transfected cells under conditions and for a time sufficient for expression of the oncogene and sufficient for cells to exhibit a transformation phenotype, wherein an observation of cell transformation indicates that the polypeptide is located in the subcellular location where the oncoprotein acts to transform the cell.

76. A kit comprising an expression vector of claim 10 in suitable packaging.

77. A kit comprising a selectable library of expression vectors of any one of claims 40, 41, 42, and 43 in suitable packaging.
